(19) United States
(12) Patent Application Publication
(10) Pub. No.: US 2015/0353908 A1
(43) Pub. Date: Dec. 10, 2015

(54) THERAPEUTICS FOR SUPPRESSING OSTEOPOROSIS

(71) Applicant: IPSEN BIOINNOVATION LIMITED, Abingdon, Oxfordshire (GB)

(72) Inventor: Keith FOSTER, Abingdon, Oxfordshire (GB)

(21) Appl. No.: 14/758,435

(22) PCT Filed: Feb. 21, 2014
(86) PCT No.: PCT/GB2014/050538

§ 371 Date: Jun. 29, 2015

(30) Foreign Application Priority Data
Feb. 21, 2013 (GB) 1303108.3

(51) Int. Cl. C12N 9/48; C07K 14/33

(52) U.S. Cl. CPC C12N 9/48; C07K 14/33; A61K 38/00

(57) ABSTRACT

The invention provides a polypeptide, for use in suppressing or treating osteoporosis, wherein the polypeptide comprises: a non-cytotoxic protease, which protease is capable of cleaving a SNARE protein in an enterochromaffin cell; a Targeting Moiety (TM) that is capable of binding to a Binding Site on an enterochromaffin cell, which Binding Site is capable of undergoing endocytosis to be incorporated into an endosome within the enterochromaffin cell, and wherein said enterochromaffin cell expresses said SNARE protein; and a translocation domain that is capable of translocating the protease from within an endosome, across the endosomal membrane and into the cytosol of the enterochromaffin cell; with the proviso that the polypeptide is not a clostridial neurotoxin (holotoxin) molecule.

The present invention provides methods and compositions for suppressing or treating osteoporosis.

Osteoporosis is a disease of low bone mass resulting in deterioration of the structure of bones. This deterioration can weaken bones to such an extent that fractures can occur with little trauma, resulting in a poor quality of life and a possible reduction in life expectancy. In osteoporosis, the bone mineral density (BMD) is reduced, bone microarchitecture deteriorates, and the amount and variety of proteins in bone are altered.

Bone tissue, which is mainly composed of collagen and calcium phosphate, is subjected to a constant breakdown and resynthesis (i.e., bone remodeling) in a process mediated by osteoblasts, which produce new bone, and osteoclasts, which destroy the bone. The activities of these cells are regulated by a number of cytokines and growth factors. The underlying mechanism in all cases of osteoporosis is an imbalance between bone resorption and bone formation. Because the patterns of reforming and resorbing bone often vary from patient to patient, it is thought several different factors account for this problem. Important chemicals (such as estrogen, testosterone, parathyroid hormone, and vitamin D) and blood factors that affect cell growth are involved with this process. Changes in levels of any of these factors can play a role in the development of osteoporosis.

Recently, it has been determined that gut-derived serotonin (GDS) plays a role in directing bone remodeling. Approximately 90% of the human body's total serotonin is synthesised by specialist endocrine cells in the gut, called enterochromaffin cells. Once released from enterochromaffin cells GDS circulates in the serum. Gut-derived serotonin (GDS) is a powerful inhibitor of osteoblast proliferation and bone formation that does not affect bone resorption. Thus modifying GDS levels is a potential means of altering the balance in bone remodelling and thus provides a means for suppressing or treating osteoporosis.

Globally, 1 in 3 women over 50 will suffer a fracture due to osteoporosis (this figure increases to 1 in 2 over 60), while 1 in 5 men over 50 will suffer a fracture due to osteoporosis; (this increases to 1 in 3 over 60). According to World Health Organization (WHO), osteoporosis is second only to cardiovascular disease as global healthcare problem. Since osteoporosis affects the elderly population which is growing, it will put a bigger burden to the healthcare systems.

Presently treatments for osteoporosis include lifestyle changes, such as diet and exercise, and medication, including calcium, vitamin D, bisphosphonates and hormone replacement therapies. However existing treatments are associated with adverse side-effects. Hence there remains a need in the art for new medicaments for suppressing or treating osteoporosis.

This need is addressed by the present invention, which solves one or more of the above-mentioned problems.

### SUMMARY OF THE INVENTION

The present invention addresses one or more of the above-mentioned problems by providing a fusion protein for use in suppressing or treating osteoporosis in a subject (e.g. patient), said fusion protein comprising:

(i) a non-cytotoxic protease, which protease is capable of cleaving a SNARE protein in an enterochromaffin cell;
(ii) a Targeting Moiety (TM) that is capable of binding to a Binding Site on an enterochromaffin cell, which Binding Site is capable of undergoing endocytosis to be incorporated into an endosome within the enterochromaffin cell, and wherein said enterochromaffin cell expresses said SNARE protein; and
(iii) a translocation domain that is capable of translocating the protease from within an endosome, across the endosomal membrane and into the cytosol of the enterochromaffin cell;
with the proviso that the polypeptide is not a clostridial neurotoxin (holotoxin) molecule.

The first aspect also embraces a corresponding method for suppressing or treating osteoporosis, said method comprising administering a therapeutically effective amount of a polypeptide of the present invention to a patient.

### DETAILED DESCRIPTION OF THE INVENTION

The polypeptide of the present invention is not a naturally-occurring clostridial neurotoxin molecule (also known as clostridial holotoxin). Clostridial holotoxin is one of the most lethal neurotoxins known to man, and, as such, has significant limitations as a therapeutic molecule. Also, in the context of suppressing osteoporosis, clostridial holotoxin is associated with undesirable off-site targeting, i.e. targeting of non-serotonin secreting cells.

In use, a polypeptide of the invention binds to an enterochromaffin cell. Thereafter, the translocation component effects transport of the protease component into the cytosol of the enterochromaffin cell. Finally, once inside, the protease inhibits the exocytic fusion process of the enterochromaffin cell by cleaving SNARE protein present in the cytosol of the enterochromaffin cell. Thus, by inactivating the exocytic fusion apparatus of the enterochromaffin cell, the polypeptide of the invention inhibits secretion of serotonin therefrom. Accordingly, the polypeptide of the invention reduces the level of serum serotonin and hence is capable of suppressing or treating osteoporosis.

The polypeptides of the present invention provide a distinct advantage over other therapeutics in that they have the potential to inhibit the secretion of serotonin from a specific target cell, i.e. enterochromaffin cells. In contrast, other proposed therapeutic agents seek to reduce serum serotonin levels by attempting to non-specifically block the synthesis of that serotonin, or to use an antagonist to the serotonin receptor that mediates the effect of serotonin. However, the present invention provides a means of specifically blocking serotonin secretion from its site of production.

The principal target cell of the present invention is an enterochromaffin cell. Enterochromaffin cells are located in the duodenum and are the source of serum serotonin.

The fusion proteins of the present invention generally demonstrate a reduced binding affinity (in the region of up to 100-fold) for target cells when compared with the corresponding ‘free’ TM (i.e. the isolated TM per se). However, despite this observation, the fusion proteins of the present invention surprisingly demonstrate good efficacy. This can be attributed to two principal features. First, the non-cytotoxic protease component is catalytic—thus, the therapeutic effect of a few such molecules is rapidly amplified within a target cell. Secondly, the receptors present on the target cells need only act as a gateway for entry of the therapeutic, and need not necessarily be stimulated to a level required in order to achieve a ligand-receptor mediated pharmacological response. Accordingly, the fusion proteins of the present invention may be administered at a dosage that is lower than would be employed for other types of therapeutic molecules, which are typically administered at high microgram to milligram (even up to hundreds of milligram) quantities. In contrast, the fusion proteins of the present invention may be administered at much lower dosages, typically at least 10-fold lower, and more typically at 100-fold lower.

### The Non-Cytotoxic Protease

The biologically active component of the TSI polypeptides of the present invention is a non-cytotoxic protease. Thus, once delivered into the cytosol of a target cell, the non-cytotoxic protease component effects SNARE cleavage within the desired target cell. Since SNARE proteins are an essential component of the secretory process within mammalian target cells, proteolytic inactivation thereof inhibits/suppresses secretion from said target cells.

Non-cytotoxic proteases are a discrete class of molecules that do not kill cells; instead, they act by inhibiting cellular processes other than protein synthesis. Non-cytotoxic proteases are produced by a variety of higher organisms (e.g. plants, and animals)—an example of such a higher organism is the Brazilian scorpion. In addition, non-cytotoxic proteases are produced by a variety of microorganisms, notably bacteria such as Clostridium sp. and Neisseria sp.

Clostridial neurotoxins represent a major group of non-cytotoxic toxin molecules, and comprise two polypeptide chains joined together by a disulphide bond. The two chains are termed the heavy chain (H-chain), which has a molecular mass of approximately 100 kDa, and the light chain (L-chain), which has a molecular mass of approximately 50 kDa. It is the L-chain, which possesses a protease function and exhibits high substrate specificity for vesicle and/or plasma membrane associated (SNARE) proteins involved in the exocytic process (e.g. synaptobrevin, syntaxin, SNAP and/or VAMP). These substrates are important components of a cell's secretory machinery.

Neisseria sp., most notably from the species N. gonorrhoeae, produce functionally similar non-cytotoxic toxin molecules. An example of such a non-cytotoxic protease is IgA protease (see WO99/58571). Similar IgA proteases are produced by streptococci, such as Streptococcus pneumoniae.

Thus, in one embodiment the non-cytotoxic protease of the present invention may be a clostridial neurotoxin protease or an IgA protease (see, for example, WO 99/032272). Another example of non-cytotoxic proteases is a scorpion venom protease, such as those from the venom of the Brazilian scorpion Tityus serrulatus, or the protease antarease (see, for example, WO 2011/022357).

### The Targeting Moiety (TM)

Turning now to the Targeting Moiety (TM) component of the present invention, it is this component that binds the polypeptide of the present invention to a enterochromaffin cell. The TM is preferably a peptide. The TM typically comprises a maximum of 50 amino acid residues, for example a maximum of 40 amino acid residues or a maximum of 30 amino acid residues or a maximum of 20 amino acid residues.

As described above, it has been determined that elevated levels of serum serotonin causes decreased bone mass, leading to osteoporosis. The enterochromaffin cells of the duodenum are the source of serum serotonin.

In use, a polypeptide of the invention binds to an enterochromaffin cell. Thereafter, the translocation component effects transport of the protease component into the cytosol of the enterochromaffin cell. Finally, once inside, the protease inhibits the exocytic fusion process of the enterochromaffin cell by cleaving SNARE protein present in the cytosol of the enterochromaffin cell. Thus, by inactivating the exocytic fusion apparatus of the enterochromaffin cell, the polypeptide of the invention inhibits secretion of serotonin therefrom. Accordingly, the polypeptide of the invention reduces the level of serum serotonin and hence is capable of suppressing or treating osteoporosis.

The TM binds to a Binding Site on the enterochromaffin cell, thereby providing selectivity of the polypeptide to this species of target cell over other cells. In this regard, preferred TM embodiments of the present invention include antibodies (eg. monoclonal antibodies, antibody fragments such as Fab, F(ab)′₂, Fv, ScFv, etc., and antibody domains peptides), as well as binding scaffolds, which bind to the receptors identified below. Accordingly, the polypeptides of present invention may include commercially available antibodies or binding scaffolds, which have been designed to achieve specific binding to the target cell or receptor in question. Alternatively, preferred TMs include peptide ligands, such as cytokines, growth factors, neuropeptides, and lectins.

A TM of the present invention binds to a receptor on an enterochromaffin cell. By way of example, a TM of the polypeptide of the present invention binds to a receptor on an enterochromaffin cell selected from the group comprising: an IL13 receptor (e.g. IL13Rα1); a somatostatin receptor (e.g. SST_{R}2 or SST_{R}5); a VPAC receptor (e.g. VPAC₁ or VPAC₂); a TGFβI receptor (e.g. TGFβRI or TGβRII); a tachykinin receptor (e.g. TAC₁ or TAC₂); a gamma-aminobutyric acid (GABA) receptor (e.g. GABA_{A} receptors, particularly α6 or β2); epidermal growth factor (EGF) receptor (e.g. EGF_{R}); fibroblast growth factor receptor (e.g. FGFᵣ2); or a peptide YY (PYY) receptor (e.g.neuropeptide Y receptor Y₁ or Y₂). All of these receptors are expressed on enterochromaffin cells.

In one embodiment, the TM is selected from: an IL13 receptor ligand (e.g. IL13); a somatostatin receptor ligand (e.g. somatostatin); a VPAC receptor ligand; a TGFβI receptor ligand (e.g. TGF βI); a tachykinin receptor ligand (e.g. substance P); a gamma-aminobutyric acid (GABA) receptor ligand; epidermal growth factor (EGF) receptor ligand (e.g. EFG; TGFα, heparin-binding EGF-like growth factor (HB-EGF), amphiregulin (AR), betacellulin (BTC), epiregulin (EPR) or epigen); fibroblast growth factor receptor ligand (e.g. FGF); or a peptide YY (PYY) receptor ligand (e.g. peptide YY); as well as truncations and peptide analogues thereof.

In one embodiment the TM of the polypeptide of the present invention binds to a receptor on an enterochromaffin cell selected from the group comprising: IL13Rα1, SST_{R}2, SST_{R}5, VPAC₁, VPAC₂, TGFβRI, TGFβRII, TAC₁, TAC₂, GABA_{A} receptor α6, GABA_{A} receptor β2, EGF_{R}, FGFᵣ2, neuropeptide Y receptor Y₁ or neuropeptide Y receptor Y₂. All of these receptors are expressed on enterochromaffin cells.

In one embodiment, the TM is selected from: an IL13 peptide, an somatostatin peptide, a VPAC peptide, a TGFβI peptide, a substance P peptide, an EGF peptide, a TGFα peptide, a heparin-binding EGF-like growth factor (HB-EGF) peptide, an amphiregulin (AR) peptide, a betacellulin (BTC) peptide, an epiregulin (EPR) peptide, an epigen peptide, a fibroblast growth factor (FGF) peptide, or peptide YY; as well as truncations and peptide analogues thereof.

In one embodiment, a TM of the polypeptide of the present invention binds to an IL13 receptor, preferably IL13Rα1. Suitable examples of such TMs include: IL13 peptides such as full length IL13 peptide (e.g IL13₁₄₆) and truncations or peptide analogues thereof.

In one embodiment, a TM of the polypeptide of the present invention binds to a somatostatin (SST) receptor. By way of example, suitable TMs include: SST peptides and cortistatin (CST)-peptides, as well as peptide analogues thereof such as D-Phe-Phe-Phe-D-Trp-Lys-Thr-Phe-Thr-NH₂ (BIM 23052), D-Phe-Phe-Tyr-D-Trp-Lys-Val-Phe-D-Nal-NH₂ (BIM 23056) or c[Cys-Phe-Phe-D-Trp-Lys-Thr-Phe-Cys]-NH₂ (BIM-23268). Further examples include the SST peptides SST-14 and SST-28; as well as peptide and peptide analogues such as: octreotide, lanreotide, BIM23027, vapreotide, seglitide, and SOM230. These TMs are preferred TMs for binding to SST receptors, in particular to SST_{R}2 and SST_{R}5 receptors.

In one embodiment, a TM of the present invention binds to a VPAC receptor, preferably VPAC₁ or VPAC₂. Suitable examples of such TMs include PACAP(1-27), or a truncation of peptide analogue thereof, including the analogue TP3805, the analogue [Arg^{15,20,21}Leu¹⁷]-PACAP-Gly-Lys-Arg-NH2, and analogue R3P66 [HSDAVFTDNYTRLRKQVAAKKYLQSIKNKR Y]. Further suitable examples of such TMs include VIP-1 and VIP-2 peptides, for example VIP(1-28), or a truncation or peptide analogue thereof, including the VIP analogues TP3939, TP4200, TP3982. These TMs demonstrate a selective binding to VPAC₁. Alternatively, a TM demonstrating a selective binding to VPAC₂ may be employed, such as, for example mROM (see Yu et al., Peptides 27 (6) p1359-66 (2006), which is hereby incorporated by reference thereto).

In one embodiment, a TM of the present invention binds to a TGFβI receptor, preferably TGFβRI or TGFβRII. Suitable examples of such TMs include TGF β peptides, preferably TGF βI peptide and truncations or peptide analogues thereof.

In one embodiment, a TM of the present invention binds to a tachykinin receptor, preferably TAC₁ or TAC₂. Suitable examples of such TMs include Substance P, truncations or peptide analogues thereof including [pGlu⁵,MePhe⁸,Sar⁹]-SP₅₋₁₁(D1Me-C7), Eledoisin Related Peptide (ERP) and (D-pro⁴, D-trp^{7,9})SP(4-11). Further suitable TMs binding a TSI to a tachykinin receptor are described in detail in WO 2011/020114, which is hereby incorporated in its entirety by reference thereto.

In one embodiment, a TM of the present invention binds to a gamma-aminobutyric acid (GABA) receptor preferably GABA receptor α6 or β2. Suitable examples of such TMs include gamma-aminobutyric acid (GABA), diazepam binding inhibitor (DBI) peptide, benzodiazepine, GABA analogues, DBI analogues.

In one embodiment, a TM of the present invention binds to an epidermal growth factor (EGF) receptor, preferably EGF_{R}. Suitable examples of such TMs include epidermal growth factor (EGF); TGFα, heparin-binding EGF-like growth factor (HB-EGF), amphiregulin (AR), betacellulin (BTC), epiregulin (EPR) or epigen, and truncations or peptide analogues thereof including LONG®EGF (a recombinant analogue of TGFα plus a 14 amino acid N-term inal extension peptide.

In one embodiment, a TM of the present invention binds to fibroblast growth factor receptor, preferably FGFᵣ2. Suitable examples of such TMs include fibroblast growth factor receptor ligand FGF as well as truncations and peptide analogues thereof, including PG-FGF-1 (a fusion of FGF with proteoglycan (PG) core protein), basic FGF ([Val¹¹²]) basic FGF (106-146)NH2). Further suitable TMs binding to a fibroblast growth factor receptor are described in detail in U.S. Pat. No. 6,294,359, which is hereby incorporated in its entirety by reference thereto.

In one embodiment, a TM of the present invention binds to peptide YY (PYY) receptor, preferably neuropeptide Y receptor Y₁ or Y₂. Suitable examples of such TMs include peptide YY as well as truncations and peptide analogues thereof, including PYY (22-36) (BIM-43004), PYY (1-36), PYY (9-36), PYY (14-36), PYY (22-36), PYY (27-36).

In a preferred embodiment, the TM binds to the IL13 receptor, preferably IL13Rα1; also preferred is that the TM is an IL13 peptide or a truncation or peptide analogue thereof.

### The Translocation Domain

The translocation component of the present invention enables translocation of the non-cytotoxic protease (or fragment thereof) into the target cell so that functional expression of protease activity occurs within the cytosol of the target cell. The translocation component is preferably capable of forming ion-permeable pores in lipid membranes (e.g. endosomal membranes) under conditions of low pH. The translocation component may be obtained from a microbial protein source, for example a bacterial or viral protein source. Hence, in one embodiment, the translocation component comprises or consists of a translocation domain of an enzyme, such as a bacterial toxin. In another embodiment, the translocation domain comprises or consists of the translocation domain of a viral protein. In one embodiment, the translocation component of the present invention may comprise or consist of a clostridial neurotoxin H-chain or a fragment thereof such as the H_{N} domain (or a translocating fragment thereof) of a clostridial neurotoxin.

### Polypeptide Preparation

The polypeptides of the present invention comprise 3 principal components: a ‘bioactive’ (ie. a non-cytotoxic protease); a TM; and a translocation domain. The general technology associated with the preparation of such fusion proteins is often referred to as re-targeted toxin technology. By way of exemplification, we refer to: WO94/21300; WO96/33273; WO98/07864; WO00/10598; WO01/21213; WO06/059093; WO00/62814; WO00/04926; WO93/15766; WO00/61192; and WO99/58571. All of these publications are herein incorporated by reference thereto.

In more detail, the TM component of the present invention may be fused to either the protease component or the translocation component of the present invention. Said fusion is preferably by way of a covalent bond, for example either a direct covalent bond or via a spacer/linker molecule. The protease component and the translocation component are preferably linked together via a covalent bond, for example either a direct covalent bond or via a spacer/linker molecule. Suitable spacer/linked molecules are well known in the art, and typically comprise an amino acid-based sequence of between 5 and 40, preferably between 10 and 30 amino acid residues in length.

In use, the polypeptides have a di-chain conformation, wherein the protease component and the translocation component are linked together, preferably via a disulphide bond.

The polypeptides of the present invention may be prepared by conventional chemical conjugation techniques, which are well known to a skilled person. By way of example, reference is made to Hermanson, G. T. (1996), Bioconjugate techniques, Academic Press, and to Wong, S. S. (1991), Chemistry of protein conjugation and cross-linking, CRC Press, Nagy et al., PNAS 95 p1794-99 (1998). Further detailed methodologies for attaching synthetic TMs to a polypeptide of the present invention are provided in, for example, EP0257742. The above-mentioned conjugation publications are herein incorporated by reference thereto.

Alternatively, the polypeptides may be prepared by recombinant preparation of a single polypeptide fusion protein (see, for example, WO98/07864). This technique is based on the in vivo bacterial mechanism by which native clostridial neurotoxin (i.e. holotoxin) is prepared, and results in a fusion protein having the following ‘simplified’ structural arrangement:

NH₂-[protease component]-[translocation component]-[TM]-COOH

According to WO98/07864, the TM is placed towards the C-terminal end of the fusion protein. The fusion protein is then activated by treatment with a protease, which cleaves at a site between the protease component and the translocation component. A di-chain protein is thus produced, comprising the protease component as a single polypeptide chain covalently attached (via a disulphide bridge) to another single polypeptide chain containing the translocation component plus TM.

Alternatively, according to WO06/059093, the TM component of the fusion protein is located towards the middle of the linear fusion protein sequence, between the protease cleavage site and the translocation component. This ensures that the TM is attached to the translocation domain (i.e. as occurs with native clostridial holotoxin), though in this case the two components are reversed in order vis-à-vis native holotoxin. Subsequent cleavage at the protease cleavage site exposes the N-terminal portion of the TM, and provides the di-chain polypeptide fusion protein.

A further alternative is the ‘split ligand’ presentation of the TM component of the fusion protein. Here the TM component, which acts as a ligand, has both a free N-terminal domain and a free C-terminal domain. Thus, the TM is capable of interacting with the binding site (e.g. a receptor or acceptor) on a target cell via an interaction between an N-terminal portion of the targeting moiety and a domain of the binding site. Alternatively, the TM is capable of an interaction between the C-terminal portion of the targeting moiety and a domain of a binding site. Or, the TM is capable of a dual interaction, wherein an N-terminal portion of the targeting moiety interacts with a domain of the binding site and a C-terminal portion of the targeting moiety interacts with a domain of a binding site. In this latter embodiment, the N- and C-terminal portions of the TM may bind to the same or different domains of a binding site, and/or may bind to domains on different binding sites. Further information regarding this arrangement may be found in WO2012/156743 which is hereby incorporated by reference thereto.

The above-mentioned protease cleavage sequence(s) may be introduced (and/or any inherent cleavage sequence removed) at the DNA level by conventional means, such as by site-directed mutagenesis. Screening to confirm the presence of cleavage sequences may be performed manually or with the assistance of computer software (e.g. the MapDraw program by DNASTAR, Inc.). Whilst any protease cleavage site may be employed (i.e. clostridial, or non-clostridial), the following are preferred:

(SEQ ID NO: 1)
Enterokinase(DDDDK↓)
(SEQ ID NOS 2 and 3)
Factor Xa (IEGR↓/IDGR↓)
(SEQ ID NO: 4)
TEV(Tobacco Etch virus)(ENLYFQ↓G)
(SEQ ID NO: 5)
Thrombin (LVPR↓GS)
(SEQ ID NO: 6)
PreScission (LEVLFQ↓GP).

Additional protease cleavage sites include recognition sequences that are cleaved by a non-cytotoxic protease, for example by a clostridial neurotoxin. These include the SNARE (e.g. SNAP-25, syntaxin, VAMP) protein recognition sequences that are cleaved by non-cytotoxic proteases such as clostridial neurotoxins. Particular examples are provided in US2007/0166332, which is hereby incorporated in its entirety by reference thereto.

Also embraced by the term protease cleavage site is an intein, which is a self-cleaving sequence. The self-splicing reaction is controllable, for example by varying the concentration of reducing agent present. The above-mentioned ‘activation’ cleavage sites may also be employed as a ‘destructive’ cleavage site (discussed below) should one be incorporated into a polypeptide of the present invention.

In a preferred embodiment, the fusion protein of the present invention may comprise one or more N-terminal and/or C-terminal located purification tags. Whilst any purification tag may be employed, the following are preferred:

His-tag (e.g. 6×histidine) (SEQ ID NO: 7), preferably as a C-terminal and/or

### N-terminal tag

MBP-tag (maltose binding protein), preferably as an N-terminal tag
GST-tag (glutathione-S-transferase), preferably as an N-terminal tag
His-MBP-tag, preferably as an N-terminal tag
GST-MBP-tag, preferably as an N-terminal tag
Thioredoxin-tag, preferably as an N-terminal tag
CBD-tag (Chitin Binding Domain), preferably as an N-terminal tag.

One or more peptide spacer/linker molecules may be included in the fusion protein. For example, a peptide spacer may be employed between a purification tag and the rest of the fusion protein molecule.

The present invention also provides a DNA sequence that encodes the above-mentioned fusion protein. In a preferred aspect of the present invention, the DNA sequence is prepared as part of a DNA vector, wherein the vector comprises a promoter and terminator.

In a preferred embodiment, the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |

The DNA construct of the present invention is preferably designed in silico, and then synthesised by conventional DNA synthesis techniques.

The above-mentioned DNA sequence information is optionally modified for codon-biasing according to the ultimate host cell (e.g. E. coli) expression system that is to be employed.

The DNA backbone is preferably screened for any inherent nucleic acid sequence, which when transcribed and translated would produce an amino acid sequence corresponding to the protease cleavage site encoded by the second peptide-coding sequence. This screening may be performed manually or with the assistance of computer software (e.g. the MapDraw program by DNASTAR, Inc.).

Reference to “suppressing” and “treating” as used herein, means to provide a therapeutic benefit to a subject. It includes, for example, administering a fusion protein as defined herein to prevent or lessen the severity of osteoporosis.

A further aspect of the invention provides a method of preventing or suppressing osteoporosis, wherein said method comprises administering to said subject a therapeutically effective amount of a fusion protein comprising:

(i) a non-cytotoxic protease, which protease is capable of cleaving a SNARE protein in an enterochromaffin cell;
(ii) a Targeting Moiety (TM) that is capable of binding to a Binding Site on an enterochromaffin cell, which Binding Site is capable of undergoing endocytosis to be incorporated into an endosome within the enterochromaffin cell, and wherein said enterochromaffin cell expresses said SNARE protein; and
(iii) a translocation domain that is capable of translocating the protease from within an endosome, across the endosomal membrane and into the cytosol of the enterochromaffin cell;
with the proviso that the polypeptide is not a clostridial neurotoxin (holotoxin) molecule.

The fusion proteins of the present invention may include a destructive protease cleavage site, which is susceptible to cleavage (by a local protease) in the event that the fusion protein might migrate to an off-site location. This approach helps to minimise the risk of off-site targeting. Thus, the fusion proteins of the present invention may be designed to include one or more destructive cleavage sites, for example, as described in WO2010/094905 and WO2002/44199—each of these documents is hereby incorporated in its entirety by reference thereto.

### Polypeptide Delivery

In use, the present invention employs a pharmaceutical composition, comprising a polypeptide, together with at least one component selected from a pharmaceutically acceptable carrier, excipient, adjuvant, propellant and/or salt.

The polypeptides of the present invention may be formulated for oral, parenteral, continuous infusion, implant, inhalation or topical application. Compositions suitable for injection may be in the form of solutions, suspensions or emulsions, or dry powders which are dissolved or suspended in a suitable vehicle prior to use.

Local delivery means may include an oral or gastric delivery. In this regard, formulations in enteric-coated capsules or other particulate systems such as microspheres can be used. Local administration to the duodenum via laparoscopic surgery is also possible. Other examples of local delivery may also include transdermal delivery (via an adhesive patch).

The preferred route of administration is selected from: systemic, oral, laparoscopic and/or localised injection.

In the case of formulations for injection, it is optional to include a pharmaceutically active substance to assist retention at or reduce removal of the polypeptide from the site of administration. One example of such a pharmaceutically active substance is a vasoconstrictor such as adrenaline. Such a formulation confers the advantage of increasing the residence time of polypeptide following administration and thus increasing and/or enhancing its effect.

The dosage ranges for administration of the polypeptides of the present invention are those to produce the desired therapeutic effect. It will be appreciated that the dosage range required depends on the precise nature of the polypeptide or composition, the route of administration, the nature of the formulation, the age of the patient, the nature, extent or severity of the patient's condition, contraindications, if any, and the judgement of the attending physician. Variations in these dosage levels can be adjusted using standard empirical routines for optimisation.

Suitable daily dosages (per kg weight of patient) are in the range 0.0001-1 mg/kg, preferably 0.0001-0.5 mg/kg, more preferably 0.002-0.5 mg/kg, and particularly preferably 0.004-0.5 mg/kg. The unit dosage can vary from less that 1 microgram to 30 mg, but typically will be in the region of 0.01 to 1 mg per dose, which may be administered daily or preferably less frequently, such as weekly or six monthly.

A particularly preferred dosing regimen is based on 2.5 ng of polypeptide as the 1× dose. In this regard, preferred dosages are in the range 1×-100× (i.e. 2.5-250 ng).

Fluid dosage forms are typically prepared utilising the polypeptide and a pyrogen-free sterile vehicle. The polypeptide, depending on the vehicle and concentration used, can be either dissolved or suspended in the vehicle. In preparing solutions the polypeptide can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving. Advantageously additives such as buffering, solubilising, stabilising, preservative or bactericidal, suspending or emulsifying agents and or local anaesthetic agents may be dissolved in the vehicle.

Dry powders, which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then freeze dried and the containers are sealed aseptically.

Parenteral suspensions, suitable for intramuscular, subcutaneous or intradermal injection, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Advantageously, a suspending agent for example polyvinylpyrrolidone is included in the composition(s) to facilitate uniform distribution of the components.

Administration in accordance with the present invention may take advantage of a variety of delivery technologies including microparticle encapsulation, viral delivery systems or high-pressure aerosol impingement.

### DEFINITIONS SECTION

Targeting Moiety (TM) means any chemical structure that functionally interacts with a Binding Site to cause a physical association between the polypeptide of the invention and the surface of a target cell. In the context of the present invention, the target cell is a enterochromaffin cell. The term TM embraces any molecule (i.e. a naturally occurring molecule, or a chemically/physically modified variant thereof) that is capable of binding to a Binding Site on the target cell, which Binding Site is capable of internalisation (eg. endosome formation)—also referred to as receptor-mediated endocytosis. The TM may possess an endosomal membrane translocation function, in which case separate TM and Translocation Domain components need not be present in an agent of the present invention. Throughout the preceding description, specific TMs have been described. Reference to said TMs is merely exemplary, and the present invention embraces all variants and derivatives thereof, which retain the basic binding (i.e. targeting) ability of the exemplified TMs.

A TM according to the present invention includes antibodies (e.g. antibody fragments) and binding scaffolds; especially commercially available antibodies/fragments and scaffolds designed for the purpose of binding (e.g. specifically) to target cells.

Protein scaffolds represent a new generation of universal binding frameworks to complement the expanding repertoire of therapeutic monoclonal antibodies and derivatives such as scFvs, Fab molecules, dAbs (single-domain antibodies), camelids, diabodies and minibodies, each of which may be employed as a TM of the present invention. Scaffold systems create or modify known protein recognition domains either through creation of novel scaffolds or modification of known protein binding domains. Such scaffolds include but are not limited to:

(i) protein A based scaffolds—affibodies (Nord, K. et al 1997 “Binding proteins selected from combinatorial libraries of an alpha-helical bacterial receptor domain”. Nat Biotechnol 15, 772-777);
(ii) lipocalin based scaffolds—anticalins (Skerra 2008 “Alternative binding proteins: anticalins—harnessing the structural plasticity of the lipocalin ligand pocket to engineer novel binding activities”. FEBS J. 275:2677-83);
(iii) fibronectin based scaffolds—adnectin (Dineen et al 2008 “The Adnectin CT-322 is a novel VEGF receptor 2 inhibitor that decreases tumor burden in an orthotopic mouse model of pancreatic cancer”. BMC Cancer 8:352);
(iv) avimers (Silverman et al 2005 “Multivalent avimer proteins evolved by exon shuffling of a family of human receptor domains”. Nat Biotechnol 23:1556-61);
(v) ankyrin based scaffolds—darpins (Zahnd et al 2006 “Selection and characterization of Her2 binding-designed ankyrin repeat proteins”. J Biol Chem. 281:35167-75); and
(vi) centyrin scaffolds—based on a protein fold that has significant structural homology to Ig domains with loops that are analogous to CDRs. Ig domains are a common module in human proteins and have been widely applied as alternative scaffold proteins. Each of the above ‘scaffold’ publications is hereby incorporated (in its entirety) by reference thereto.

Binding scaffolds can be used to target particular cell types via interaction with specific cell surface proteins, receptors or other cell surface epitopes such as sugar groups. Such modified scaffolds can be engineered onto recombinant non-cytotoxic protease based polypeptides of the present invention.

The TM of the present invention binds (preferably specifically binds) to the enterochromaffin target cell in question. The term “specifically binds” preferably means that a given TM binds to the target cell with a binding affinity (Ka) of 10⁶ M⁻¹ or greater, preferably 10⁷ M⁻¹ or greater, more preferably 10⁸ M⁻¹ or greater, and most preferably, 10⁹ M⁻¹ or greater. The term “specifically binds” can also mean that a given TM binds to a given receptor, IL13 receptor (e.g. IL13Rα1); a somatostatin receptor (e.g. SST_{R}2 or SST_{R}5); a VPAC receptor (e.g. VPAC₁ or VPAC₂); a TGFβI receptor (e.g. TGFβRI or TGFβRII); a tachykinin receptor (e.g. TAC₁ or TAC₂); a gamma-aminobutyric acid (GABA) receptor (e.g. GABA_{A} receptors, particularly α6 or β2); epidermal growth factor (EGF) receptor (e.g. EGF_{R}); fibroblast growth factor receptor (e.g. FGFᵣ2); or a peptide YY (PYY) receptor (e.g. neuropeptide Y receptor Y₁ or Y₂) with a binding affinity (Ka) of 10⁶ M⁻¹ or greater, preferably 10⁷ M⁻¹ or greater, more preferably 10⁸ M⁻¹ or greater, and most preferably, 10⁹ M⁻¹ or greater.

Reference to TM in the present specification embraces fragments and variants thereof, which retain the ability to bind to the target cell in question. By way of example, a variant may have at least 80%, preferably at least 90%, more preferably at least 95%, and most preferably at least 97 or at least 99% amino acid sequence homology with the reference TM (e.g. any SEQ ID NO presented in the present specification, which defines a TM). Thus, a variant may include one or more analogues of an amino acid (e.g. an unnatural amino acid), or a substituted linkage. Also, by way of example, the term fragment, when used in relation to a TM, means a peptide having at least ten, preferably at least twenty, more preferably at least thirty, and most preferably at least forty amino acid residues of the reference TM. The term fragment also relates to the above-mentioned variants. Thus, by way of example, a fragment of the present invention may comprise a peptide sequence having at least 10, 20, 30 or 40 amino acids, wherein the peptide sequence has at least 80% sequence homology over a corresponding peptide sequence (of contiguous) amino acids of the reference peptide.

It is routine to confirm that a TM binds to the selected target cell. For example, a simple radioactive displacement experiment may be employed in which tissue or cells representative of a target cell in question are exposed to labelled (e.g. tritiated) TM in the presence of an excess of unlabelled TM. In such an experiment, the relative proportions of non-specific and specific binding may be assessed, thereby allowing confirmation that the TM binds to the target cell. Optionally, the assay may include one or more binding antagonists, and the assay may further comprise observing a loss of TM binding. Examples of this type of experiment can be found in Hulme, E. C. (1990), Receptor-binding studies, a brief outline, pp. 303-311, In Receptor biochemistry, A Practical Approach, Ed. E. C. Hulme, Oxford University Press.

In the context of the present invention, reference to a peptide TM embraces peptide analogues thereof, so long as the analogue binds to the same receptor as the corresponding ‘reference’ TM. Said analogues may include synthetic residues such as:

β-Nal=β-naphthylalanine
β-Pal=β-pyridylalanine
hArg(Bu)=N-guanidino-(butyl)-homoarginine
hArg(Et)₂=N, N′-guanidino-(dimethyl)-homoarginine
hArg(CH₂CF₃)₂=N, N′-guanidino-bis-(2,2,2,-trifluoroethyl)-homoarginine
hArg(CH₃, hexyl)=N, N′-guanidino-(methyl, hexyl)-homoarginine
Lys(Me)=N^{e}-methyllysine
Lys(iPr)=N^{e}-isopropyllysine
AmPhe=aminomethylphenylalanine
AChxAla=aminocyclohexylalanine
Abu=α-aminobutyric acid
Tpo=4-thiaproline

### MeLeu=N-methylleucine

Orn=ornithine
Nle—norleucine
Nva=norvaline
Trp(Br)=5-bromo-tryptophan
Trp(F)=5-fluoro-tryptophan
Trp(NO₂)=5-nitro-tryptophan
Gaba=γ-aminobutyric acid

### Bmp=J-mercaptopropionyl

Ac=acetyl
Pen—pencillamine

The fusion proteins (also referred to herein as polypeptides) of the present invention may lack a functional H_{C} or H_{CC} domain of a clostridial neurotoxin. In one embodiment, the polypeptides lack the last 50 C-terminal amino acids of a clostridial neurotoxin holotoxin. In another embodiment, the polypeptides lack the last 100, 150, 200, 250, or 300 C-terminal amino acid residues of a clostridial neurotoxin holotoxin. Alternatively, the H_{C} binding activity may be negated/reduced by mutagenesis—by way of example, referring to BoNT/A for convenience, modification of one or two amino acid residue mutations (W1266 to L and Y1267 to F) in the ganglioside binding pocket causes the H_{C} region to lose its receptor binding function. Analogous mutations may be made to non-serotype A clostridial peptide components, e.g. a construct based on botulinum B with mutations (W1262 to L and Y1263 to F) or botulinum E (W1224 to L and Y1225 to F). Other mutations to the active site achieve the same ablation of H_{C} receptor binding activity, e.g. Y1267S in botulinum type A toxin and the corresponding highly conserved residue in the other clostridial neurotoxins. Details of this and other mutations are described in Rummel et al (2004) (Molecular Microbiol. 51:631-634), which is hereby incorporated by reference thereto.

The H_{C} peptide of a native clostridial neurotoxin comprises approximately 400-440 amino acid residues, and consists of two functionally distinct domains of approximately 25 kDa each, namely the N-terminal region (commonly referred to as the H_{CN} peptide or domain) and the C-terminal region (commonly referred to as the H_{CC} peptide or domain). Moreover, it has been well documented that the C-terminal region (H_{CC}), which constitutes the C-terminal 160-200 amino acid residues, is responsible for binding of a clostridial neurotoxin to its natural cell receptors, namely to nerve terminals at the neuromuscular junction. Thus, reference throughout this specification to a clostridial heavy-chain lacking a functional heavy chain H_{C} peptide (or domain) such that the heavy-chain is incapable of binding to cell surface receptors to which a native clostridial neurotoxin binds means that the clostridial heavy-chain simply lacks a functional H_{CC} peptide. In other words, the H_{CC} peptide region is either partially or wholly deleted, or otherwise modified (e.g. through conventional chemical or proteolytic treatment) to inactivate its native binding ability for nerve terminals at the neuromuscular junction.

Thus, in one embodiment, a clostridial H_{N} peptide of the present invention lacks part of a C-terminal peptide portion (H_{CC}) of a clostridial neurotoxin and thus lacks the H_{C} binding function of native clostridial neurotoxin. By way of example, in one embodiment, the C-terminally extended clostridial H_{N} peptide lacks the C-terminal 40 amino acid residues, or the C-terminal 60 amino acid residues, or the C-terminal 80 amino acid residues, or the C-terminal 100 amino acid residues, or the C-terminal 120 amino acid residues, or the C-terminal 140 amino acid residues, or the C-terminal 150 amino acid residues, or the C-terminal 160 amino acid residues of a clostridial neurotoxin heavy-chain. In another embodiment, the clostridial H_{N} peptide of the present invention lacks the entire C-terminal peptide portion (H_{CC}) of a clostridial neurotoxin and thus lacks the H_{C} binding function of native clostridial neurotoxin. By way of example, in one embodiment, the clostridial H_{N} peptide lacks the C-terminal 165 amino acid residues, or the C-terminal 170 amino acid residues, or the C-terminal 175 amino acid residues, or the C-terminal 180 amino acid residues, or the C-terminal 185 amino acid residues, or the C-terminal 190 amino acid residues, or the C-terminal 195 amino acid residues of a clostridial neurotoxin heavy-chain. By way of further example, the clostridial H_{N} peptide of the present invention lacks a clostridial H_{CC} reference sequence selected from the group consisting of:
Botulinum type A neurotoxin—amino acid residues (Y1111-L1296)
Botulinum type B neurotoxin—amino acid residues (Y1098-E1291)
Botulinum type C neurotoxin—amino acid residues (Y1112-E1291)
Botulinum type D neurotoxin—amino acid residues (Y1099-E1276)
Botulinum type E neurotoxin—amino acid residues (Y1086-K1252)
Botulinum type F neurotoxin—amino acid residues (Y1106-E1274)
Botulinum type G neurotoxin—amino acid residues (Y1106-E1297)
Tetanus neurotoxin—amino acid residues (Y1128-D1315).

The above-identified reference sequences should be considered a guide as slight variations may occur according to sub-serotypes.

The protease of the present invention embraces all non-cytotoxic proteases that are capable of cleaving one or more proteins of the exocytic fusion apparatus in eukaryotic cells.

The protease of the present invention is preferably a bacterial protease (or fragment thereof). More preferably the bacterial protease is selected from the genera Clostridium or Neisseria/Streptococcus (e.g. a clostridial L-chain, or a neisserial IgA protease preferably from N. gonorrhoeae or S. pneumoniae). Another example of non-cytotoxic proteases includes scorpion venom protease, such as those from the venom of the Brazilian scorpion Tityus serrulatus, or the protease antarease.

The present invention also embraces variant non-cytotoxic proteases (i.e. variants of naturally-occurring protease molecules), so long as the variant proteases still demonstrate the requisite protease activity. By way of example, a variant may have at least 70%, preferably at least 80%, more preferably at least 90%, and most preferably at least 95% or at least 98% amino acid sequence homology with a reference protease sequence. Thus, the term variant includes non-cytotoxic proteases having enhanced (or decreased) endopeptidase activity—particular mention here is made to the increased K_{cat}/Kₘ of BoNT/A mutants Q161A, E54A, and K165L see Ahmed, S. A. (2008) Protein J. DOI 10.1007/s10930-007-9118-8, which is incorporated by reference thereto. The term fragment, when used in relation to a protease, typically means a peptide having at least 150, preferably at least 200, more preferably at least 250, and most preferably at least 300 amino acid residues of the reference protease. As with the TM ‘fragment’ component (discussed above), protease ‘fragments’ of the present invention embrace fragments of variant proteases based on a reference sequence.

The protease of the present invention preferably demonstrates a serine or metalloprotease activity (e.g. endopeptidase activity). The protease is preferably specific for a SNARE protein (e.g. SNAP-25, synaptobrevin/VAMP, or syntaxin).

Particular mention is made to the protease domains of neurotoxins, for example the protease domains of bacterial neurotoxins. Thus, the present invention embraces the use of neurotoxin domains, which occur in nature, as well as recombinantly prepared versions of said naturally-occurring neurotoxins.

Exemplary neurotoxins are produced by clostridia, and the term clostridial neurotoxin embraces neurotoxins produced by C. tetani (TeNT), and by C. botulinum (BoNT) serotypes A-G, as well as the closely related BoNT-like neurotoxins produced by C. baratii and C. butyricum. The above-mentioned abbreviations are used throughout the present specification. For example, the nomenclature BoNT/A denotes the source of neurotoxin as BoNT (serotype A). Corresponding nomenclature applies to other BoNT serotypes.

BoNTs are the most potent toxins known, with median lethal dose (LD50) values for mice ranging from 0.5 to 5 ng/kg depending on the serotype. BoNTs are adsorbed in the gastrointestinal tract, and, after entering the general circulation, bind to the presynaptic membrane of cholinergic nerve terminals and prevent the release of their neurotransmitter acetylcholine. BoNT/B, BoNT/D, BoNT/F and BoNT/G cleave synaptobrevin/vesicle-associated membrane protein (VAMP); BoNT/C, BoNT/A and BoNT/E cleave the synaptosomal-associated protein of 25 kDa (SNAP-25); and BoNT/C cleaves syntaxin.

BoNTs share a common structure, being di-chain proteins of ˜150 kDa, consisting of a heavy chain (H-chain) of ˜100 kDa covalently joined by a single disulphide bond to a light chain (L-chain) of ˜50 kDa. The H-chain consists of two domains, each of ˜50 kDa. The C-terminal domain (H_{C}) is required for the high-affinity neuronal binding, whereas the N-terminal domain (H_{N}) is proposed to be involved in membrane translocation. The L-chain is a zinc-dependent metalloprotease responsible for the cleavage of the substrate SNARE protein.

The term L-chain fragment means a component of the L-chain of a neurotoxin, which fragment demonstrates a metalloprotease activity and is capable of proteolytically cleaving a vesicle and/or plasma membrane associated protein involved in cellular exocytosis.

Examples of suitable protease (reference) sequences include:
Botulinum type A neurotoxin—amino acid residues (1-448)
Botulinum type B neurotoxin—amino acid residues (1-440)
Botulinum type C neurotoxin—amino acid residues (1-441)
Botulinum type D neurotoxin—amino acid residues (1-445)
Botulinum type E neurotoxin—amino acid residues (1-422)
Botulinum type F neurotoxin—amino acid residues (1-439)
Botulinum type G neurotoxin—amino acid residues (1-441)
Tetanus neurotoxin—amino acid residues (1-457)
IgA protease—amino acid residues (1-959)*
* Pohlner, J. et al. (1987). Nature 325, pp. 458-462, which is hereby incorporated by reference thereto.

The above-identified reference sequence should be considered a guide as slight variations may occur according to sub-serotypes. By way of example, US 2007/0166332 (hereby incorporated by reference thereto) cites slightly different clostridial sequences:
Botulinum type A neurotoxin—amino acid residues (M1-K448)
Botulinum type B neurotoxin—amino acid residues (M1-K441)
Botulinum type C neurotoxin—amino acid residues (M1-K449)
Botulinum type D neurotoxin—amino acid residues (M1-R445)
Botulinum type E neurotoxin—amino acid residues (M1-R422)
Botulinum type F neurotoxin—amino acid residues (M1-K439)
Botulinum type G neurotoxin—amino acid residues (M1-K446)
Tetanus neurotoxin—amino acid residues (M1-A457)

A variety of clostridial toxin fragments comprising the light chain can be useful in aspects of the present invention with the proviso that these light chain fragments can specifically target the core components of the neurotransmitter release apparatus and thus participate in executing the overall cellular mechanism whereby a clostridial toxin proteolytically cleaves a substrate. The light chains of clostridial toxins are approximately 420-460 amino acids in length and comprise an enzymatic domain. Research has shown that the entire length of a clostridial toxin light chain is not necessary for the enzymatic activity of the enzymatic domain. As a non-limiting example, the first eight amino acids of the BoNT/A light chain are not required for enzymatic activity. As another non-limiting example, the first eight amino acids of the TeNT light chain are not required for enzymatic activity. Likewise, the carboxyl-terminus of the light chain is not necessary for activity. As a non-limiting example, the last 32 amino acids of the BoNT/A light chain (residues 417-448) are not required for enzymatic activity. As another non-limiting example, the last 31 amino acids of the TeNT light chain (residues 427-457) are not required for enzymatic activity. Thus, aspects of this embodiment can include clostridial toxin light chains comprising an enzymatic domain having a length of, for example, at least 350 amino acids, at least 375 amino acids, at least 400 amino acids, at least 425 amino acids and at least 450 amino acids. Other aspects of this embodiment can include clostridial toxin light chains comprising an enzymatic domain having a length of, for example, at most 350 amino acids, at most 375 amino acids, at most 400 amino acids, at most 425 amino acids and at most 450 amino acids.

Further examples of suitable non-cytotoxic proteases are described in detail in WO2007/106115, which is hereby incorporated in its entirety by reference thereto.

In one embodiment, the non-cytotoxic protease cleaves a non-neuronal SNARE protein such as a SNAP-23 protein. In one embodiment, the non-cytotoxic protease is a modified botulinum toxin L-chain capable of cleaving SNAP-23. An example of such a modified L-chain is described by Chen and Barbieri, PNAS, vol. 106, no. 23, p9180-9184, 2009.

In one embodiment, the non-cytotoxic protease is a BoNT/A, BoNT/C or BoNT/E protease, and the preferred SNARE motif is a SNAP (e.g. SNAP 25) motif.

In another embodiment, the non-cytotoxic protease is a BoNT/B, BoNT/D, BoNT/F or BoNT/G or tetanus neurotoxin (TeNT) protease, and the preferred SNARE motif is a VAMP motif.

In another embodiment, the non-cytotoxic protease is a BoNT/Cᵢ protease, and the preferred SNARE motif is a syntaxin motif.

The non-cytotoxic proteases of the present invention recognise different cleavage site sequences and thus have slightly different cleavage specificities.

| | Cleavage site recognition sequence: | | | | | | |
|---|---|---|---|---|---|---|---|
| Non-cytotoxic | P4-P3-P2-P1-↓-P1′-P2′-P3′ | | | | | | |
| Protease | P4 | P3 | P2 | P1 | P1′ | P2′ | P3′ |
| BoNT/A | E | A | N | Q | R | A | T |
| BoNT/B | G | A | S | Q | F | E | T |
| BoNT/C | A | N | Q | R | A | T | K |
| BoNT/C | D | T | K | K | A | V | K |
| BoNT/D | R | D | Q | K | L | S | E |
| BoNT/E | Q | I | D | R | I | M | E |
| BoNT/F | E | R | D | Q | K | L | S |
| BoNT/G | E | T | S | A | A | K | I |
| TeNT | G | A | S | Q | F | E | T |
| IgA protease | S | T | P | P | T | P | S |
| Antarease | I | K | R | K | Y | W | W |

By way of further example, reference is made to the following recognition sequences and cleavage sites:

| Non- | Cleavage site recognition sequence: | | | | | | |
|---|---|---|---|---|---|---|---|
| cytotoxic | P4-P3-P2-P1-↓-P1′-P2′-P3′ | | | | | | |
| Protease | P4 | P3 | P2 | P1 | P1′ | P2′ | P3′ |
| BoNT/A | E | A | N | Q | R | A | T |
| | A | N | Q | R | A | T | K |
| | E | A | N | Q | R | A | T |
| | F | A | N | Q | R | A | T |
| | E | A | N | Q | R | A | T |
| | E | A | N | Q | R | A | I |
| | E | A | N | K | A | T | K |
| | E | A | N | K | H | A | T |
| | E | A | N | K | H | A | N |
| | | | | Q | R | | |
| | | | | K | H | | |
| BoNT/C | D | E | A | N | Q | R | A |
| | E | A | N | Q | R | A | T |
| | A | N | Q | R | A | T | K |
| | N | Q | R | A | T | K | M |
| | A | N | Q | R | A | I | K |
| | A | N | Q | R | A | H | Q |
| | D | T | K | K | A | V | K |
| | K | T | K | K | A | V | K |
| | E | T | K | K | A | I | K |
| | E | T | K | R | A | M | K |
| | D | T | K | K | A | V | R |
| | D | T | K | K | A | L | K |
| | D | T | K | K | A | M | K |
| | E | S | K | K | A | V | K |
| | E | T | K | K | A | M | K |
| | E | T | K | K | A | V | K |
| | | | | K | A | | |
| | | | | R | A | | |
| BoNT/E | Q | I | D | R | I | M | E |
| | Q | I | Q | K | I | T | E |
| | Q | I | D | R | I | V | E |
| | Q | F | D | R | I | M | D |
| | Q | F | D | R | I | M | E |
| | Q | L | D | R | I | H | D |
| | Q | I | D | R | I | M | D |
| | Q | V | D | R | I | Q | Q |
| | | | | R | I | | |
| | | | | K | I | | |
| BoNT/B | G | A | S | Q | F | E | T |
| | A | G | A | S | Q | F | E |
| | G | A | S | Q | F | E | S |
| | Q | A | S | Q | F | E | S |
| | G | A | S | Q | G | E | T |
| | G | A | S | Q | F | E | Q |
| | Q | A | S | Q | F | E | A |
| | G | A | S | Q | F | Q | Q |
| | G | A | S | Q | F | E | A |
| | | | | Q | F | | |
| BoNT/D | R | D | Q | K | L | S | E |
| | R | D | Q | K | I | S | E |
| | K | D | Q | K | L | A | E |
| | | | | K | L | | |
| BoNT/F | E | R | D | Q | K | L | S |
| | V | L | E | R | D | Q | K |
| | E | R | D | Q | K | I | S |
| | E | R | D | Q | A | L | S |
| | E | K | D | Q | K | L | A |
| | | | | Q | K | | |
| BoNT/G | E | S | S | A | A | K | I |
| | E | T | S | A | A | K | I |
| | E | S | S | A | A | K | L |
| | E | T | S | A | A | K | L |
| | | | | A | A | | |
| TeNT | G | A | S | Q | F | E | T |
| | G | A | S | Q | G | E | T |
| | G | A | S | Q | F | E | Q |
| | Q | A | S | Q | F | E | A |
| | G | A | S | Q | F | E | S |
| | Q | A | S | Q | F | E | S |
| | G | A | S | Q | F | Q | Q |
| | G | A | S | Q | F | E | A |
| | | | | Q | F | | |
| IgA | S | T | P | P | T | P | S |
| protease | | | | | | | |
| Antarease | I | K | R | K | Y | W | W |

The polypeptides of the present invention, especially the protease component thereof, may be PEGylated—this may help to increase stability, for example duration of action of the protease component. PEGylation is particularly preferred when the protease comprises a BoNT/A, B or C₁ protease. PEGylation preferably includes the addition of PEG to the N-terminus of the protease component. By way of example, the N-terminus of a protease may be extended with one or more amino acid (e.g. cysteine) residues, which may be the same or different. One or more of said amino acid residues may have its own PEG molecule attached (e.g. covalently attached) thereto. An example of this technology is described in WO2007/104567, which is incorporated in its entirety by reference thereto.

A Translocation Domain is a molecule that enables translocation of a protease into a target cell such that a functional expression of protease activity occurs within the cytosol of the target cell. Whether any molecule (e.g. a protein or peptide) possesses the requisite translocation function of the present invention may be confirmed by any one of a number of conventional assays.

For example, Shone C. (1987) describes an in vitro assay employing liposomes, which are challenged with a test molecule. Presence of the requisite translocation function is confirmed by release from the liposomes of K⁺ and/or labelled NAD, which may be readily monitored [see Shone C. (1987) Eur. J. Biochem; vol. 167(1): pp. 175-180].

A further example is provided by Blaustein R. (1987), which describes a simple in vitro assay employing planar phospholipid bilayer membranes. The membranes are challenged with a test molecule and the requisite translocation function is confirmed by an increase in conductance across said membranes [see Blaustein (1987) FEBS Letts; vol. 226, no. 1: pp. 115-120].

Additional methodology to enable assessment of membrane fusion and thus identification of Translocation Domains suitable for use in the present invention are provided by Methods in Enzymology Vol 220 and 221, Membrane Fusion Techniques, Parts A and B, Academic Press 1993.

The present invention also embraces variant translocation domains, so long as the variant domains still demonstrate the requisite translocation activity. By way of example, a variant may have at least 70%, preferably at least 80%, more preferably at least 90%, and most preferably at least 95% or at least 98% amino acid sequence homology with a reference translocation domain. The term fragment, when used in relation to a translocation domain, means a peptide having at least 20, preferably at least 40, more preferably at least 80, and most preferably at least 100 amino acid residues of the reference translocation domain. In the case of a clostridial translocation domain, the fragment preferably has at least 100, preferably at least 150, more preferably at least 200, and most preferably at least 250 amino acid residues of the reference translocation domain (eg. H_{N} domain). As with the TM ‘fragment’ component (discussed above), translocation ‘fragments’ of the present invention embrace fragments of variant translocation domains based on the reference sequences.

The Translocation Domain is preferably capable of formation of ion-permeable pores in lipid membranes under conditions of low pH. Preferably it has been found to use only those portions of the protein molecule capable of pore-formation within the endosomal membrane.

The Translocation Domain may be obtained from a microbial protein source, in particular from a bacterial or viral protein source. Hence, in one embodiment, the Translocation Domain is a translocating domain of an enzyme, such as a bacterial toxin or viral protein.

It is well documented that certain domains of bacterial toxin molecules are capable of forming such pores. It is also known that certain translocation domains of virally expressed membrane fusion proteins are capable of forming such pores. Such domains may be employed in the present invention.

The Translocation Domain may be of a clostridial origin, such as the H_{N} domain (or a functional component thereof). H_{N} means a portion or fragment of the H-chain of a clostridial neurotoxin approximately equivalent to the amino-terminal half of the H-chain, or the domain corresponding to that fragment in the intact H-chain. In this regard, should it be desired to remove the H_{C} cell-binding function, this may be done by deletion of the H_{C} or H_{CC} amino acid sequence (either at the DNA synthesis level, or at the post-synthesis level by nuclease or protease treatment). Alternatively, the H_{C} function may be inactivated by chemical or biological treatment.

Examples of suitable (reference) Translocation Domains include:
Botulinum type A neurotoxin—amino acid residues (449-871)
Botulinum type B neurotoxin—amino acid residues (441-858)
Botulinum type C neurotoxin—amino acid residues (442-866)
Botulinum type D neurotoxin—amino acid residues (446-862)
Botulinum type E neurotoxin—amino acid residues (423-845)
Botulinum type F neurotoxin—amino acid residues (440-864)
Botulinum type G neurotoxin—amino acid residues (442-863)
Tetanus neurotoxin—amino acid residues (458-879)

The above-identified reference sequence should be considered a guide as slight variations may occur according to sub-serotypes. By way of example, US 2007/0166332 (hereby incorporated by reference thereto) cites slightly different clostridial sequences:
Botulinum type A neurotoxin—amino acid residues (A449-K871)
Botulinum type B neurotoxin—amino acid residues (A442-S858)
Botulinum type C neurotoxin—amino acid residues (T450-N866)
Botulinum type D neurotoxin—amino acid residues (D446-N862)
Botulinum type E neurotoxin—amino acid residues (K423-K845)
Botulinum type F neurotoxin—amino acid residues (A440-K864)
Botulinum type G neurotoxin—amino acid residues (5447-5863)
Tetanus neurotoxin—amino acid residues (5458-V879)

Further examples of suitable translocation domains are described in detail in WO2007/106115, which is hereby incorporated in its entirety by reference thereto.

In the context of the present invention, a variety of clostridial toxin H_{N} regions comprising a translocation domain can be useful in aspects of the present invention with the proviso that these active fragments can facilitate the release of a non-cytotoxic protease (e.g. a clostridial L-chain) from intracellular vesicles into the cytoplasm of the target cell and thus participate in executing the overall cellular mechanism whereby a clostridial toxin proteolytically cleaves a substrate. The H_{N} regions from the heavy chains of clostridial toxins are approximately 410-430 amino acids in length and comprise a translocation domain. Research has shown that the entire length of a H_{N} region from a clostridial toxin heavy chain is not necessary for the translocating activity of the translocation domain. Thus, aspects of this embodiment can include clostridial toxin H_{N} regions comprising a translocation domain having a length of, for example, at least 350 amino acids, at least 375 amino acids, at least 400 amino acids and at least 425 amino acids. Other aspects of this embodiment can include clostridial toxin H_{N} regions comprising translocation domain having a length of, for example, at most 350 amino acids, at most 375 amino acids, at most 400 amino acids and at most 425 amino acids.

For further details on the genetic basis of toxin production in Clostridium botulinum and C. tetani, we refer to Henderson et al (1997) in The Clostridia: Molecular Biology and Pathogenesis, Academic press.

The term H_{N} embraces naturally-occurring neurotoxin H_{N} portions, and modified H_{N} portions having amino acid sequences that do not occur in nature and/or synthetic amino acid residues, so long as the modified H_{N} portions still demonstrate the above-mentioned translocation function.

Alternatively, the Translocation Domain may be of a non-clostridial origin. Examples of non-clostridial (reference) Translocation Domain origins include, but not be restricted to, the translocation domain of diphtheria toxin [O=Keefe et al., Proc. Natl. Acad. Sci. USA (1992) 89, 6202-6206; Silverman et al., J. Biol. Chem. (1993) 269, 22524-22532; and London, E. (1992) Biochem. Biophys. Acta., 1112, pp. 25-51], the translocation domain of Pseudomonas exotoxin type A [Prior et al. Biochemistry (1992) 31, 3555-3559], the translocation domains of anthrax toxin [Blanke et al. Proc. Natl. Acad. Sci. USA (1996) 93, 8437-8442], a variety of fusogenic or hydrophobic peptides of translocating function [Plank et al. J. Biol. Chem. (1994) 269, 12918-12924; and Wagner et al (1992) PNAS, 89, pp. 7934-7938], and amphiphilic peptides [Murata et al (1992) Biochem., 31, pp. 1986-1992]. The Translocation Domain may mirror the Translocation Domain present in a naturally-occurring protein, or may include amino acid variations so long as the variations do not destroy the translocating ability of the Translocation Domain.

Particular examples of viral (reference) Translocation Domains suitable for use in the present invention include certain translocating domains of virally expressed membrane fusion proteins. For example, Wagner et al. (1992) and Murata et al. (1992) describe the translocation (i.e. membrane fusion and vesiculation) function of a number of fusogenic and amphiphilic peptides derived from the N-terminal region of influenza virus haemagglutinin. Other virally expressed membrane fusion proteins known to have the desired translocating activity are a translocating domain of a fusogenic peptide of Semliki Forest Virus (SFV), a translocating domain of vesicular stomatitis virus (VSV) glycoprotein G, a translocating domain of SER virus F protein and a translocating domain of Foamy virus envelope glycoprotein. Virally encoded Aspike proteins have particular application in the context of the present invention, for example, the E1 protein of SFV and the G protein of the G protein of VSV.

Use of the (reference) Translocation Domains listed in Table (below) includes use of sequence variants thereof. A variant may comprise one or more conservative nucleic acid substitutions and/or nucleic acid deletions or insertions, with the proviso that the variant possesses the requisite translocating function. A variant may also comprise one or more amino acid substitutions and/or amino acid deletions or insertions, so long as the variant possesses the requisite translocating function.

| Translocation | Amino acid | |
|---|---|---|
| Domain source | residues | References |
| Diphtheria | 194-380 | Silverman et al., 1994, J. Biol. |
| toxin | | Chem. 269, 22524-22532 |
| | | London E., 1992, Biochem. |
| | | Biophys. Acta., 1113, 25-51 |
| Domain II of | 405-613 | Prior et al., 1992, |
| pseudomonas | | Biochemistry 31, 3555-3559 |
| exotoxin | | Kihara & Pastan, 1994, |
| | | Bioconj Chem. 5, 532-538 |
| Influenza virus | GLFGAIAGFIENGW | Plank et al., 1994, J. Biol. |
| haemagglutinin | EGMIDGWYG, and | Chem. 269, 12918-12924 |
| | Variants thereof | Wagner et al., 1992, PNAS, |
| | | 89, 7934-7938 |
| | | Murata et al., 1992, |
| | | Biochemistry 31, 1986-1992 |
| Semliki Forest | Translocation domain | Kielian et al., 1996, J Cell Biol. |
| virus fusogenic | | 134(4), 863-872 |
| protein | | |
| Vesicular | 118-139 | Yao et al., 2003, Virology |
| Stomatitis virus | | 310(2), 319-332 |
| glycoprotein G | | |
| SER virus F | Translocation domain | Seth et al., 2003, J Virol |
| protein | | 77(11) 6520-6527 |
| Foamy virus | Translocation domain | Picard-Maureau et al., 2003, J |
| envelope | | Virol. 77(8), 4722-4730 |
| glycoprotein | | |

The polypeptides of the present invention may further comprise a translocation facilitating domain. Said domain facilitates delivery of the non-cytotoxic protease into the cytosol of the target cell and are described, for example, in WO08/008803 and WO08/008805, each of which is herein incorporated by reference thereto.

By way of example, suitable translocation facilitating domains include an enveloped virus fusogenic peptide domain, for example, suitable fusogenic peptide domains include influenza virus fusogenic peptide domain (eg. influenza A virus fusogenic peptide domain of 23 amino acids), alphavirus fusogenic peptide domain (eg. Semliki Forest virus fusogenic peptide domain of 26 amino acids), vesiculovirus fusogenic peptide domain (eg. vesicular stomatitis virus fusogenic peptide domain of 21 amino acids), respirovirus fusogenic peptide domain (eg. Sendai virus fusogenic peptide domain of 25 amino acids), morbiliivirus fusogenic peptide domain (eg. Canine distemper virus fusogenic peptide domain of 25 amino acids), avulavirus fusogenic peptide domain (eg. Newcastle disease virus fusogenic peptide domain of 25 amino acids), henipavirus fusogenic peptide domain (eg. Hendra virus fusogenic peptide domain of 25 amino acids), metapneumovirus fusogenic peptide domain (eg. Human metapneumovirus fusogenic peptide domain of 25 amino acids) or spumavirus fusogenic peptide domain such as simian foamy virus fusogenic peptide domain; or fragments or variants thereof.

By way of further example, a translocation facilitating domain may comprise a Clostridial toxin H_{CN} domain or a fragment or variant thereof. In more detail, a Clostridial toxin H_{CN} translocation facilitating domain may have a length of at least 200 amino acids, at least 225 amino acids, at least 250 amino acids, at least 275 amino acids. In this regard, a Clostridial toxin H_{CN} translocation facilitating domain preferably has a length of at most 200 amino acids, at most 225 amino acids, at most 250 amino acids, or at most 275 amino acids. Specific (reference) examples include:
Botulinum type A neurotoxin—amino acid residues (872-1110)
Botulinum type B neurotoxin—amino acid residues (859-1097)
Botulinum type C neurotoxin—amino acid residues (867-1111)
Botulinum type D neurotoxin—amino acid residues (863-1098)
Botulinum type E neurotoxin—amino acid residues (846-1085)
Botulinum type F neurotoxin—amino acid residues (865-1105)
Botulinum type G neurotoxin—amino acid residues (864-1105)
Tetanus neurotoxin—amino acid residues (880-1127)

The above sequence positions may vary a little according to serotype/sub-type, and further examples of suitable (reference) Clostridial toxin H_{CN} domains include:
Botulinum type A neurotoxin—amino acid residues (874-1110)
Botulinum type B neurotoxin—amino acid residues (861-1097)
Botulinum type C neurotoxin—amino acid residues (869-1111)
Botulinum type D neurotoxin—amino acid residues (865-1098)
Botulinum type E neurotoxin—amino acid residues (848-1085)
Botulinum type F neurotoxin—amino acid residues (867-1105)
Botulinum type G neurotoxin—amino acid residues (866-1105)
Tetanus neurotoxin—amino acid residues (882-1127)

Any of the above-described facilitating domains may be combined with any of the previously described translocation domain peptides that are suitable for use in the present invention. Thus, by way of example, a non-clostridial facilitating domain may be combined with non-clostridial translocation domain peptide or with clostridial translocation domain peptide. Alternatively, a Clostridial toxin H_{CN} translocation facilitating domain may be combined with a non-clostridal translocation domain peptide. Alternatively, a Clostridial toxin H_{CN} facilitating domain may be combined or with a clostridial translocation domain peptide, examples of which include:
Botulinum type A neurotoxin—amino acid residues (449-1110)
Botulinum type B neurotoxin—amino acid residues (442-1097)
Botulinum type C neurotoxin—amino acid residues (450-1111)
Botulinum type D neurotoxin—amino acid residues (446-1098)
Botulinum type E neurotoxin—amino acid residues (423-1085)
Botulinum type F neurotoxin—amino acid residues (440-1105)
Botulinum type G neurotoxin—amino acid residues (447-1105)
Tetanus neurotoxin—amino acid residues (458-1127)

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

### Alignment Scores for Determining Sequence Identity

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{[ \begin{matrix}length of the longer sequence plus the \\ number of gaps introduced into the longer \\ sequence in order to align the two sequences\end{matrix} ]} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic: arginine
lysine
histidine
   Acidic: glutamic acid
aspartic acid
   Polar: glutamine
asparagine
   Hydrophobic: leucine
isoleucine
valine
   Aromatic: phenylalanine
tryptophan
tyrosine
   Small: glycine
alanine
serine
threonine
methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for clostridial polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxyproline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenised polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

In one embodiment, the TM does not substantially bind to a VIP receptor and/or to a CRH receptor.

In one embodiment, the TM is not PACAP (1-38) or the VIP peptide, or a CRH peptide.

There now follows a brief description of the Figures, which illustrate aspects and/or embodiments of the present invention.

### EXAMPLES

A polypeptide is prepared for administration to a patient suffering from osteoporosis, providing a method for the suppression or treatment of osteoporosis. As defined in the present invention, the Targeting Moiety (TM) component of the prepared polypeptide (specifically) binds to a receptor present on the enterochromaffin cell. Thereafter, the translocation component effects transport of the non-cytotoxic protease component of the polypeptide, which once inside the enterochromaffin cell, inhibits serotonin secretion therefrom. Accordingly, the polypeptide reduces the level of serum serotonin, and is capable of suppressing or treating osteoporosis.

### Example 1

A post-menopausal 60 year-old female patient with a stooped posture, suffers from debilitating chronic pain attributed to multiple vertebral fractures. Her partner recently passed away, and she is struggling to care for herself independently. Following hormone replacement therapy (HRT), she experiences significant weight gain and resulting reduced mobility. Treatment is by subcutaneous injection of 0.025 mg/kg of a polypeptide of the present invention comprising an IL13₁₄₆ peptide ligand (weekly injection over a period of six months). Inhibition of serotonin release leads to an increase in bone strength and a marked improvement in posture. The patient reports effective pain relief and no further fragility fractures.

### Example 2

A 60 year-old male patient reports chronic back pain and presents a severe case of the classical Dowager's hump. The patient works as a farmer but has not been able to tend his farm for over three months due to his worsening condition. A parenteral suspension of 0.07 mg/kg of a polypeptide of the present invention comprising a cortistatin (CST)-peptide analogue (D-Phe-Phe-Phe-D-Trp-Lys-THr-Phe-TH-NH₂ [BIM-23268]) is administered via laparoscopic duodenal injection (6 monthly injection regimen). Positive results including reduced back pain are reported within the first month of treatment.

### Example 3

An 87 year-old female patient with a family history of osteoporosis has suffered five fractures in her ribs and one fracture in her wrist in a period of two months. The fractures are having a significant effect on her physical well-being and quality of life. Weekly dosage of 0.09 mg/kg of a polypeptide of the present invention comprising TP3805 peptide is administered intravenously. No further fractures are reported by the patient's doctor throughout the treatment period.

### Example 4

A 30 year-old asthmatic female patient receives inhaled-glucocorticoid medication prednisone (7.5 mg). She has been receiving the medication for a prolonged period (over 25 years) as a second-line treatment to her severe asthma condition. Recently the patient has shown possible glucocorticoid-induced-osteoporosis symptoms (analogous to Cushing's Syndrome), and breaks her wrist after a light-impact fall in the gym. Her physician prescribes a monthly dosage of 0.015 mg/kg of a polypeptide of the invention comprising TGFβ1, via intravenous injection. During the six months of treatment, the patient reports no further bone breakages.

### Example 5

A 65 year-old female patient reports two bone fractures in her wrists following a fall whilst gardening. The patient discloses that she emigrated from northern Latvia ten years ago, and has a medical history of rickets. In addition to vitamin D and calcium supplements, the patient is prescribed monthly intravenous injections of 0.05 mg/kg of a polypeptide of the invention comprising urocortin. The patient reports an improved rate of recovery, and no further bone injury after three months of treatment.

### Example 6

A 16 year-old female patient with Anorexia Nervosa collapses on her way down stairs. The patient breaks three ribs and both wrists. The patient's menstrual cycle has already ceased in a condition known as amenorrhea, which is common at the latter stages of the disease. A classical follow-on is osteoporosis, where bone density is reduced. The patient is prescribed a monthly oral dose of 0.1 mg/kg of an encapsulated formulation of a polypeptide of the invention comprising Substance P. The patient reports improvement in her condition after treatment.

### Example 7

A 77 year-old lactose-intolerant male patient falls whilst walking his dog. The patient breaks both wrists and suffers acute pain. A conventional radiographic scan shows thin bone density, and the patient is diagnosed with osteoporosis. The clinician prescribes 0.001 mg/kg (local laproscopic injection every 8 weeks) of a polypeptide of the invention comprising a targeting moiety which binds GABA receptor α6 and inhibits secretion of gut-serotonin into the blood. After two months the clinician reports good progress suggesting bone strengthening.

### Example 8

An 88 year-old female patient has suffered with osteoporosis for three years, and endured multiple bone fractures in her wrist, rib and leg. Since diagnosis, the patient has experienced a reduced overall quality of life, including an upsetting move into an elderly care-facility. The patient is given treatment for her osteoporosis with monthly injections of 0.07 mg/kg of a polypeptide of the present invention comprising epiregulin. During the six month treatment program, the patient reports no further bone fractures/injury.

### Example 9

A 59 year-old male patient with severe rheumatoid arthritis has subsequently developed osteoporosis due to inflammation of the synovium around his knee-joint. The inflammation has caused damage to the knee bone, with low bone density seen in a radiographic scan, and recurrent knee fractures. The patient's physician prescribes 0.1 mg/kg of a polypeptide of the invention comprising PF-FGF-1, administered by laparoscopic duodenal injection every three months. After one month of treatment, the patient reports no further fractures.

### Example 10

A 94 year-old male patient with osteoporosis has suffered numerous fractures in his wrist and hip. Treatment by surgery in his condition, and mature age is not recommended. However, treatment or suppression of osteoporosis by transdermal delivery of a polypeptide of the invention does not present the same risk factors to the patient. The patient is treated by administering an adhesive patch onto the surface of his skin, which delivers the polypeptide via slow diffusion from the patch over a period of 2 weeks. The transdermal patch is replaced every 12 weeks and no further fractures are reported.

### Animal Model Examples

The polypeptides of the invention are tested as a treatment for low bone-mass diseases, namely osteoporosis, through the use of ovariectomized rodents. As seen clinically in postmenopausal women, this disease model shows a higher increase in bone resorption than the increase in bone formation.

### Example 11

Six week old female C57BL6/J mice sham-operated or ovariectomized are fed once daily with a polypeptide of the invention at doses ranging from 1 to 250 mg per kg body weight per day from day 1 to day 28 after ovariectomy.

The results show that mice treated with 250, 100 or even 10 mg per kg body weight per day of the polypeptide have a higher bone mass than control ovariectomized mice, and can prevent the development of ovariectomy-induced osteoporosis in mice.

### Example 12

In order to ascertain whether a polypeptide of the invention can also rescue existing ovariectomy-induced osteopenia, sham-operated or ovariectomized 6-week-old mice left without treatment for only 2 weeks are treated with a daily dose of the polypeptide (250 mg per kg body weight per day) for 4 weeks. Control ovariectomized mice develop the expected osteopenia secondary to an increase in bone resorption parameters. These parameters are also increased in polypeptide-treated ovariectomized mice; however, in these mice the increase in bone formation parameters is of such amplitude that it normalises their bone mass. Serum serotonin concentrations are decreased by 80%, but brain serotonin content was unaffected in polypeptide-treated mice.

### Example 13

Example 12 is repeated but the 6-week-old ovariectomized mice are left untreated for 6 weeks before treating them with a polypeptide of the invention at either 25, 100 or 250 mg per kg body weight per day for another 6 weeks. The polypeptide, by increasing bone formation parameters, reverses the deleterious effects of ovariectomy on bone mass and increases it to levels similar to (25 mg per kg body weight per day) or higher than (100 or 250 mg per kg body weight per day) those seen in sham-operated mice. This increase in bone mass affects vertebrae and long bones and is also present in non-ovariectomized mice. There is no change in bone length or width in any of the treatment groups. These results establish that the polypeptide can rescue, through a bone anabolic mechanism, ovariectomy-induced osteoporosis in mice even when given at a low dose (25 mg per kg body weight per day) and late after ovariectomy.

### Example 14

The rat is the rodent model of choice for postmenopausal osteoporosis because it replicates several key features of the oestrogen-deficient adult human skeleton, such as a bone loss accompanied by an increase in bone turnover rate and a specific oestrogen-preventable cancellous osteopenia. Likewise, intermittent injections of a parathyroid hormone (PTH) are the standard to which any new bone anabolic agent must be compared.

Hence, ovariectomized rats at 12 weeks of age are left untreated for 3 or 12 more weeks so that they develop a severe osteopenia. Then sham-operated or ovariectomized rats are treated for 4 weeks with either a relatively high dose of a PTH (80 μg per kg body weight per day, subcutaneously) 10 or increasing amounts of a polypeptide of the invention (25, 100 or 250 mg per kg body weight per day, orally).

Analysis of vertebrae shows that the polypeptide fully rescues the ovariectomy-induced osteopenia in rats, regardless of whether it is given 3 or 12 weeks after ovariectomy, and that it is efficacious even at the lowest dose used (25 mg per kg body weight per day).

### Example 15

Bone quality is assessed by subjecting femur and vertebra samples from untreated, PTH-treated and polypeptide-treated ovariectomized rats to a three-point bending test and a compression analysis to determine maximal load and stiffness, two surrogates of bone quality. Both parameters are decreased after ovariectomy and restored to values seen in sham-operated rats by PTH and a polypeptide of the invention (250 mg per kg body weight per day) treatments. Thus, daily oral administration of the polypeptide can revert the bone loss and architectural deterioration in rats in a dose-dependent manner. Release of serotonin from isolated human primary gastric glands to measure inhibition by fusion proteins of the invention

### Example 16

Gastric glands are isolated from endoscopic pinch biopsies of the pyloric antral mucosa as described by Wroblewski et al. (2003) J. Cell Sci., 116, 3017-3026. The biopsies are cut into small pieces of approximately 2 mm2 using a razor blade. The tissue segments are washed 3 times in Hank's balanced salt solution (HBSS) warmed to 37° C. and incubated in 5 ml of 1 mM dithiothreitol (DTT) in HBSS for 15 minutes, continuously gassing with 5% CO₂/95% O₂ at 37° C. and shaking at 100 cycles per minute. The tissue is then washed in 3 changes of HBSS (37° C.) and enzymatically dissociated by incubating in 5 ml of 0.5 mg·ml−1 collagenase A in HBSS for 30 minutes, followed by washing again in HBSS (37° C., 3 times) and a further 30-minute collagenase digestion (5 ml, 0.5 mg·ml−1). At this stage the tissue is triturated using a wide mouthed pipette and larger tissue fragments are allowed to settle for 45 seconds under gravity. The supernatant is then removed and transferred to a clean ice-chilled universal tube. The tube is vigorously shaken to release additional gastric glands and left to sediment on ice for 45 minutes. The supernatant, which contains debris, is removed with care and discarded. The remainder, which contains isolated glands, is gently mixed and equally divided into each well of tissue culture plates. Glands from biopsies of one individual patient are divided between 2 wells of 6-well cell culture plates for serotonin release studies.

Primary human gastric glands are cultured in Dulbecco's Modified Eagle's Medium Nutrient Mixture F-12 Ham (DMEM/F-12) supplemented with 10% v/v FBS, 1% v/v antibiotic-antimycotic solution, 1% v/v penicillin-streptomycin solution and 1% v/v L-glutamine solution (200 mM) at 37° C. in a humidified atmosphere of 5% CO2/95% O2. Glands are allowed to adhere overnight after the isolation and the medium is changed daily.

Glands are pre-incubated with the fusion proteins of the invention overnight before assessing the inhibition of serotonin secretion. To stimulate serotonin secretion, Ca2+ (0.5-10 mM) is added to the primary human gastric glands for 10 minutes at 37° C. Control glands are treated with 1 mM EGTA instead of Ca2+. Stimulation is terminated by quickly cooling with ice-cold medium. Serotonin and its metabolite, 5-hydroxyindole acetic acid, are extracted from both the tissue and the medium and measured by reverse-phase HPLC with electrochemical detection. The amounts of secreted serotonin (defined as that present in the medium) are normalized to the tissue serotonin content. To estimate the Ca2+-induced serotonin secretion, the normalized serotonin content of the control medium is subtracted from that in the medium from cells subjected to stimulation. The percentage inhibition of secretion by the fusion proteins of the invention is calculated as:

(Secretion from glands not pre-treated overnight with the fusion proteins of the invention)−(Secretion from glands pre-treated overnight with the fusion proteins of the invention)/(Secretion from glands not pre-treated overnight with the fusion proteins of the invention)×100.

1. A polypeptide, for use in suppressing or treating osteoporosis, wherein the polypeptide comprises:
(i) a non-cytotoxic protease selected from a clostridial neurotoxin L-chain and an IgA protease, which protease is capable of cleaving a SNARE protein in an enterochromaffin cell;
(ii) a Targeting Moiety that is capable of binding to a receptor selected from the group comprising: a VPAC receptor; a TGFβI receptor; a gamma-aminobutyric acid (GABA) receptor; fibroblast growth factor receptor; and a peptide YY (PYY) receptor on an enterochromaffin cell, which receptor is capable of undergoing endocytosis to be incorporated into an endosome within the enterochromaffin cell, and wherein said enterochromaffin cell expresses said SNARE protein; and
(iii) a translocation domain comprising a clostridial neurotoxin translocation domain that is capable of translocating the protease from within an endosome, across the endosomal membrane and into the cytosol of the enterochromaffin cell;
with the proviso that the polypeptide is not a clostridial neurotoxin (holotoxin) molecule.

2. The polypeptide according to claim 1, wherein the TM binds to a receptor selected from the group consisting of: VPAC₁, VPAC₂, TGFβRI, TGFβRII, GABA_{A} receptor α6, GABA_{A} receptor β2, FGFᵣ2, neuropeptide Y receptor Y₁ and neuropeptide Y receptor Y₂.

3. The polypeptide according to claim 1, wherein the TM is selected from the group consisting of: a TGFβI peptide, a diazepam binding inhibitor (DBI) peptide, a TGFα peptide, a heparin-binding EGF-like growth factor (HB-EGF) peptide, an amphiregulin (AR) peptide, a betacellulin (BTC) peptide, an epiregulin (EPR) peptide, an epigen peptide, a fibroblast growth factor (FGF) peptide, and peptide YY.

4. A nucleic acid encoding a polypeptide according to claim 1.

5. The polypeptide according to claim 2, wherein the TM is selected from the group consisting of: a TGFβI peptide, a diazepam binding inhibitor (DBI) peptide, a TGFα peptide, a heparin-binding EGF-like growth factor (HB-EGF) peptide, an amphiregulin (AR) peptide, a betacellulin (BTC) peptide, an epiregulin (EPR) peptide, an epigen peptide, a fibroblast growth factor (FGF) peptide, and peptide YY.

6. A nucleic acid encoding a polypeptide according to claim 2.

7. A nucleic acid encoding a polypeptide according to claim 3.

8. A nucleic acid encoding a polypeptide according to claim 5.

9. The polypeptide according to claim 1, wherein the non-cytotoxic protease is a clostridial neurotoxin L-chain.

10. A method of suppressing or treating osteoporosis, the method comprising administering to a patient in need thereof a therapeutically effective amount of a polypeptide, wherein the polypeptide comprises:
(i) a non-cytotoxic protease selected from a clostridial neurotoxin L-chain and an IgA protease, which protease is capable of cleaving a SNARE protein in an enterochromaffin cell;
(ii) a Targeting Moiety that is capable of binding to a receptor selected from the group comprising: a VPAC receptor; a TGFβI receptor; a gamma-aminobutyric acid (GABA) receptor; fibroblast growth factor receptor; and a peptide YY (PYY) receptor on an enterochromaffin cell, which receptor is capable of undergoing endocytosis to be incorporated into an endosome within the enterochromaffin cell, and wherein said enterochromaffin cell expresses said SNARE protein; and
(iii) a translocation domain comprising a clostridial neurotoxin translocation domain that is capable of translocating the protease from within an endosome, across the endosomal membrane and into the cytosol of the enterochromaffin cell;
with the proviso that the polypeptide is not a clostridial neurotoxin (holotoxin) molecule.

11. The method of claim 10, wherein the TM binds to a receptor selected from the group consisting of: VPAC₁, VPAC₂, TGFβRI, TGFβRII, GABA_{A} receptor α6, GABA_{A} receptor β2, FGFᵣ2, neuropeptide Y receptor Y₁ and neuropeptide Y receptor Y₂.

12. The method of claim 10, wherein the TM is selected from the group consisting of: a TGFβI peptide, a diazepam binding inhibitor (DBI) peptide, a TGFα peptide, a heparin-binding EGF-like growth factor (HB-EGF) peptide, an amphiregulin (AR) peptide, a betacellulin (BTC) peptide, an epiregulin (EPR) peptide, an epigen peptide, a fibroblast growth factor (FGF) peptide, and peptide YY.


SEQUENCE LISTING (7 sequences)

SEQ ID NO: 1 - protein, 5 aa, Artificial Sequence
  Synthetic peptide
DDDDK

SEQ ID NO: 2 - protein, 4 aa, Artificial Sequence
  Synthetic peptide
IEGR

SEQ ID NO: 3 - protein, 4 aa, Artificial Sequence
  Synthetic peptide
IDGR

SEQ ID NO: 4 - protein, 7 aa, Artificial Sequence
  Synthetic peptide
ENLYFQG

SEQ ID NO: 5 - protein, 6 aa, Artificial Sequence
  Synthetic peptide
LVPRGS

SEQ ID NO: 6 - protein, 8 aa, Artificial Sequence
  Synthetic peptide
LEVLFQGP

SEQ ID NO: 7 - protein, 6 aa, Artificial Sequence
  Synthetic peptide
HHHHHH